(19) United States
(12) Patent Application Publication
(10) Pub. No.: US 2005/0226823 A1
(43) Pub. Date: Oct. 13, 2005

(54) Film-shaped preparations with improved chemical stability containing active substances and method for the production thereof

(76) Inventors: Markus Krumme, MECKENHEIM (DE); Walter Muller, Andernach (DE); Christian von Falkenhausen, Meckenheim (DE)

Correspondence Address: D Peter Hochberg Company, 6th Floor, 1940 East 6th Street, Cleveland, OH, 44114, US

(21) Appl. No.: 10/517,087

(22) PCT Filed: May 8, 2003
(86) PCT No.: PCT/EP03/04816

§ 371 Date: Dec. 6, 2004

(30) Foreign Application Priority Data
Jun. 4, 2002 (DE) 102 24 612.2

(51) Int. Cl. A61K 7/20

(52) U.S. Cl. 424/53

(57) ABSTRACT

A film-like, active substance-containing preparation for application in the oral cavity or for transmucosal application. The preparation has a maximum peroxide number that is 40.

### CROSS REFERENCE TO RELATED APPLICATIONS

This application is a National Stage application of International Application No. PCT/EP03/04816, filed on May 8, 2003, which claims priority of German application numbers 102 24 612.2, filed on Jun. 4, 2002.

### BACKGROUND OF THE INVENTION

1. Field of the Invention

The invention relates to active substance-containing film-like preparations which can be used for application in the oral cavity and for transmucosal administration of active substances and have an improved chemical stability. The invention further relates to methods of manufacture by which active substance-containing films of the present invention can be obtained.

2. Description of the Prior Art

Active substance-containing films typically have a matrix which contain one or more polymers as base substances. In this polymer matrix, there is contained at least one active substance, e.g. a medicinal agent, in dissolved or dispersed form. Frequently, various further auxiliaries or additives are added to the matrix, for example in order to adjust various physical or pharmaceutical parameters. Film-shaped preparations of this kind are capable of releasing the active substances contained therein at the site of application, e.g. in the oral cavity, so that they can be absorbed by the body.

Many pharmaceutical substances are chemically unstable and can break down into degradation products, for instance due to extended storage. This degradation reaction causes a decrease of the initially present amount of active substance, and the degradation products formed can be toxicologically risky. As a consequence of this degradative reaction, the stability of the pharmaceutical products is diminished.

Certain physical parameters (e.g. temperature, action of light) can cause this degradation reaction. The chemical composition of the pharmaceutical composition can also cause this degradation reaction.

Degradation reactions to be mentioned here are, above all, oxidative reactions. These occur with particular intensity when active oxygen is present, e.g. in the presence of peroxides. These reactions are autoxidation processes, which take place as chain reactions. Such auto-oxidation reactions and the underlying reaction mechanisms are basically known to those skilled in the art. With film-shaped preparations, in particular, one has to reckon with unwanted oxidative processes as the said preparations have a relatively large surface which may be exposed to the attack of atmospheric oxygen.

To suppress such degradation reactions, these administration forms are usually produced and packed under absence of oxygen, e.g. in a nitrogen atmosphere, or one employs antioxidants.

It has, however, turned out that despite these precautionary measures a relatively strong decrease in the active substance content occurs when film-like active substance-containing preparations are stored over an extended period, especially if oxidation-sensitive active substances are concerned.

The task underlying the present invention was therefore to indicate film-like active substance-containing preparations of the type mentioned in the introductory portion of claim 1 which possess improved active substance stability. The task was further to indicate processes that enable the production of such preparations.

### SUMMARY OF THE INVENTION

Surprisingly, this task is solved by preparations and processes of manufacture according to the present claims.

According to the invention, in the film-like preparations mentioned, the active substance stability can be improved by adjusting the peroxide number of the preparation during manufacture to a value of maximally 40, preferably to not more than 15, and especially to not more than 5.

### BRIEF DESCRIPTION OF THE DRAWING

FIG. 1 is a bar graph showing the results of a stability test including examples of the preparations of the present invention.

### DETAILED DESCRIPTION OF THE PRESENT INVENTION

The peroxide number is a measure for the content of peroxides. It indicates the amount of milli-equivalents of active oxygen per kg of a substance. Due to the restriction of the peroxide number to a maximum value of 40, preferably 15, and especially 5, the inventive film-like preparations are substantially free from active oxygen.

The term “active oxygen” means oxygen which has an oxidation state greater than −2. The term in particular comprises molecular oxygen as well as peroxides of the general structure R—O—O—R′, wherein R and R′ are H atoms, or R is an alkyl residue and R′ an H atom, or R and R′ are alkyl residues, and R and R′ may be either identical or different from each other.

It has been shown that an acceptable storage stability lasting for months can only be achieved if the relative amount of the active oxygen does not exceed the value of 2%-wt. For example, an active substance-containing film-like preparation (“wafer”) may contain 200 mg of an active substance with a molecular weight of 250 dalton, which correspond to 0.8 mMol of active substance. In this case, the portion of active oxygen must not exceed 2% of that value, i.e. a content of 0.016 mMol of active oxygen must not be exceeded. This value corresponds to a peroxide number of ca. 30. The above wafer may be, for instance, a suckable wafer having a weight per area of 500 g/m² and an active substance load of 40% and having a surface dimension of 10 cm².

In the case of thinner, quickly releasing wafers with a lower active substance load, a correspondingly lower upper limit for the content of active oxygen results. If the above-mentioned active substance (molecular weight 250 dalton) is contained in an amount of 14 mg in a wafer, this corresponds to 0.056 mMol of active substance. Consequently, the content of active oxygen must not exceed the value of 0.001 mMol (corresponding to 2%). This is equivalent to a peroxide number of approx. 14. The mentioned wafer may, for example, have a weight per area of 70 g/m² and an active substance load of 20%-wt., the dead weight of a single system (wafer) being 70 mg.

There are several known methods of determining the peroxide number.
(A) The most widely used is that of reacting a defined amount of the substance to be tested in a chloroform-glacial acetic acid solution having an excess of iodide ions and subsequent retitration of the iodide formed using sodium sulfate.
(B) Less frequently used and restricted to aqueous solution is the method of reacting the substance to be tested with titanium (IV) ions and photometric determination of the peroxocomplex forming.
(C) A particularly simple method is that of a semi-quantitative peroxide test using commercially available test strips.

The invention is based on the finding that the raw materials or formulation constituents used in the production of the film-like preparations, in their initial state, frequently contain relatively high concentrations of hydro-peroxides and peroxides. This is frequently true of polymers, solvents and certain additives (e.g. permeation enhancers). Favoured by the presence of atmospheric oxygen and heavy metal impurities, radical chain reactions occur in the course of which certain bonds in the active substance molecules are attacked, e.g. C—H bonds in benzyl or allyl position, tertiary C—H bonds, and C—H bonds in the vicinity of ether oxygen atoms. Active substance molecules which contain such molecules are particularly prejudiced by peroxide attacks.

By means of the proposed reduction of the peroxide number in the preparation, the active substance instability caused by the radical chain reaction can be suppressed much more effectively than is possible with the known measures —addition of antioxidants, nitrogen atmosphere. The reason for this is presumably that the addition of antioxidants or stabilisers remains without effect in those cases where already the raw materials used, that is, the components of the formulation contain relatively high concentrations of peroxides or hydroperoxide radicals.

In principle, the peroxide number of the film-like preparations produced can be determined according to the above-described methods (A) or (B). On the other hand, it can possibly prove difficult to dissolve a sufficient amount of the film-like preparation to be tested in a not-too-large amount of the above-mentioned solvent.

For simplification, to one embodiment of the present invention determines the peroxide content of each formulation component of the film-like composition individually (for example according to one of the above-indicated methods), and subsequently calculates the peroxide number of the composition, the peroxide numbers of the individual formulation components being weighted according to their percentage in the composition and finally added together. This sum constitutes the total peroxide number of the composition. When calculating the total peroxide number, the peroxide content of the solvents used in the production must also be taken into consideration.

The calculation of the total peroxide number is illustrated by the following calculation example:

A film-like composition consists of three formulation components X, Y and Z, wherein the portion of X is 70%-wt., the portion of Y is 20%-wt., and the portion of Z is 10%-wt. For component X a peroxide number of 10 was determined, in the cases of Y and Z, the peroxide number is 15 and 30, respectively.

The total peroxide number is calculated as follows:

$$( 10 \times 70 / 100 ) + ( 15 \times 20 / 100 ) + ( 30 \times 10 / 100 ) = 7 + 3 + 3 = 13$$

As can be seen, the peroxide numbers of the individual components are weighted with a factor that corresponds to their percentage in the composition.

To further improve the stability of the active substances contained in the film-like compositions, it is provided, according to one embodiment, that the composition contain at least one antioxidant. Substances taken into consideration are, in particular, antioxidants such as ascorbic acid, ascorbyl palmitate, sodium sulfite, sodium disulfite, sodium metabisulfite, thioglycerol, thioglycol acid, tocopherols (vitamin E), tocopherol acetate, vitamin A, propyl gallate, octyl gallate, butylhydroxyanisol and butylhydroxytoluene. Apart from these, there are also many antioxidants used in the food industry which are suitable. The concentration of these substances preferably is 0.001 to 5%-wt., especially preferred 0.01 to 3%-wt., each related to the film-like composition.

The film-like compositions according to the invention are provided with a polymer matrix which can be mono-layered or multi-layered. In any case, at least one layer contains active substance. The polymer matrix contains at least one polymer, or a polymer mixture, as base substance(s). The polymer portion preferably amounts to 10 to 95%-wt., especially preferred 25-85%-wt., in each case relative to the complete film-like composition.

The thickness of the inventive active substance-containing films is preferably in the range of from 0.01 to 5 mm, especially preferred in the range of from 0.05 to 1 mm.

For making the polymer matrix, the following polymers are particularly preferred: cellulose ether, especially ethyl cellulose, propyl cellulose, carboxymethyl cellulose, hydroxypropyl cellulose, hydroxypropylmethyl cellulose, mixtures of cellulose ethers, as well as cellulose acetate, polyvinyl alcohols, polyvinyl acetate, polyvinyl pyrrolidone, polyethylene oxide polymers, polyurethane, polyacrylic acid, polyacrylates, polymethacrylates, alginates, pectins, gelatine, starch and natural rubbers.

An additional embodiment of the invention provides that the matrix contain one or more polymer(s), selected from the group of the hydrophile, water-soluble polymers or polymers degradable in aqueous media. In this way it is possible to control the active substance release from the preparation by way of the solubility or the degradability in aqueous media, e.g. in body fluids. The films can, for instance, be formulated as quickly or as slowly releasing systems.

As hydrophilic, water-soluble polymers or polymers degradable in aqueous media, the following are, in particular, to be taken into consideration: cellulose derivatives, especially hydroxypropylmethyl cellulose, carboxymethyl cellulose, hydroxypropyl cellulose and methyl cellulose, as well as polyvinyl alcohol, polyvinyl acetate, polyvinyl pyrrolidone, polyacrylates, water-soluble polysaccharides, especially pullulan, xanthan, alginates, dextranes and pectins, proteins, preferably gel-forming proteins, especially gelatine.

It is further provided, according to another embodiment, that at least one layer, or at least one surface of the preparation, have mucoadhesive properties.

The mucoadhesive properties are determined essentially by the type of the matrix-forming polymer(s) as well as by the relative portions of these polymers in the preparation. As matrix-forming polymers which may be components of a mucoadhesive formulation according to the invention, the following polymers are preferably taken into consideration—without excluding any other suitable raw materials: polyvinyl alcohols (e.g. MOWIOL®); cellulose derivatives such as hydroxpropylmethyl cellulose, hydroxypropyl cellulose, sodium carboxymethyl cellulose (e.g. Walocel), methyl cellulose, hydroxyethyl cellulose and hydroxypropylethyl cellulose; starch and starch derivatives; gelatine (various types); polyvinylpyrrolidone; gum arabic; pullulan, acrylates.

As active substances contained in the inventive preparations, basically all pharmaceutical active substances are taken into consideration, as well as any other active substances which are suitable for intervening in physiological processes in humans or animals.

The inventive preparations are especially suited for administering active substances that, due to their chemical structure, are sensitive to an increased extent to oxidative degradation reactions. Among these are those active substances which possess one of the following partial structures:
secondary or tertiary amino groups
C═C double bonds, conjugated double bonds
C—H groups in allyl position
benzylic C—H groups
tertiary C—H groups
sulfide groups, thioether groups or sulfoxide groups.

Examples for such active substances are: steroids such as 17-beta-estradiol, heterocyclic compounds such as dihydropyridine (e.g. calcium antagonists of the dihydropyridine type), nicotine, (−)-5,6,7,8,-tetrahydro-6-[propyl[2-(2-thienyl)-ethyl]amino]-1-naphthol, aromatic compounds, especially substituted aromatic compounds (e.g. adrenaline, salicylic acid and salicylic acid derivatives, phenothiazine); oxidation-sensitive biopolymers, proteins, oxidation-sensitive substances such as amine, hydroxylamine, alcohols and aldehydes.

To achieve certain effects or to modulate the chemical or physical properties, it can be advantageous if the film-like preparations contain one or more additives selected from the groups of softeners, dyes and pigments, degradation promoters, wetting agents, absorption- or permeation-enhancing substances, pH regulators, fillers, flavouring and aromatic substances and sweeteners. Pharmaceutically acceptable substances are known to those skilled in the art. These additives may preferably be present in a total concentration of up to 50%-wt, especially at a total concentration of from 1.0 to 15%-wt.

As softeners, there are, for example, those from the group of hydrocarbons, alcohols (especially higher alcohols such as dodecanol, undecanol, octanol), triglycerides, multivalent alcohols, carboxylic acids, derivatives of carboxylic acids, ethers, esters (e.g. diethyl phthalate, n-butyl adipate, citric acid esters) and amines which are taken into consideration.

To improve the physical properties, the active substance matrix may contain fillers, for example titanium dioxide, zinc oxide, chalk, active charcoal, finely dispersed silicon dioxide or corn starch.

As absorption or permeation accelerators (enhancers) those substances are especially suited which are selected from the group comprising the following substances and classes of substances: saturated or unsaturated fatty acids, fatty acid esters, especially esters with methanol, ethanol or isopropanol (e.g. oleic acid ethyl ester, oleic acid methyl ester, lauric acid methyl ester, lauric acid ethyl ester, adipic acid methyl ester, adipic acid ethyl ester), strait-chain or branched fatty alcohols or the esters thereof, especially esters with acetic acid or lactic acid (e.g. ethyl oleate, ethyl laurate, ethyl palmitate, ethyl lactate, propyl lactate, propyl palmitate, propyl laurate, propyl oleate), multivalent aliphatic alcohols or polyethylene glycols, sorbitan fatty acid esters and their derivatives obtainable by way of ethoxylation, fatty alcohol ethoxylates, polyoxyethylene fatty acid esters; lauric acid diethanolamide, oleic acid diethanolamide, coconut fatty acid diethanolamide, D-alpha-tocopherol, lauric acid hexyl ester, 2-octyl dodecanol, dexpanthenol, isopropylidene glycerol, transcutol (=diethylene glycol monoethyl ether), DEET (=N,N-diethyl-m-toluene amide), solketal, ethanol, 1,2-propanediol or other short-chain alcohols (e.g. alcohols with up to 6 C atoms), as well as menthol and other essential oils or components of essential oils. To optimize the active substance flow, it is also possible to use two or more enhancers in combination.

The inventive preparations are advantageously suitable for transmucosal administration of drugs, for example via the oral mucosa, but also to other mucosal surfaces of the body. Because of the mucoadhesive properties of the active agent-containing film layer, it is possible for a controlled active substance release to take place over a prolonged period of time. The film-like preparations can preferably be used for releasing active substances or other substances, e.g. flavouring or aromatic substances, in the oral cavity.

The inventive preparations can, in addition, be used as oral administration forms which enable the release and/or absorption of pharmaceutical active substances in the gastro-intestinal tract. In the case of suckable film-like preparations, for example, the active substance-containing solution or suspension being formed during the action of sucking can be swallowed and subsequently absorbed in the gastrointestinal tract. As suckable active substance-containing systems, relatively thick films are preferred, preferably of a thickness of up to 5 mm, especially from 0.5 to 5 mm.

The invention does, however, also comprise oral film-like administration forms which are intended for swallowing and where the active substance release substantially begins to take place only upon entering the gastrointestinal tract. This also includes such film-like active substance-containing systems which after oral administration initially disintegrate in the oral cavity into fragments, which are then swallowed.

The present invention further relates to processes of manufacture through which film-like preparations of the afore-mentioned kind can be obtained.

According to the invention, the manufacture takes place in such a manner that in a first step the peroxide number of each and every one of the formulation components intended for making the preparation according to recipe (including the solvents used) is determined.

Subsequently, in a further step, the formulation components are selected in such a manner that the sum of the peroxide numbers of the individual formulation components amounts to 40 at the most, with the peroxide number of each one of the formulation component being weighted according to the percentage of that component in the preparation.

From the formulation components thus selected, a solution, dispersion or melt is prepared which contains the active substance(s) to be released.

This solution, dispersion or melt is coated by knife coating, roll coating, spraying and extrusion methods onto an inert support, and dried or allowed to cool, which results in the formation of a film layer.

If during the determination of the peroxide numbers of the individual components it turns out that the peroxide content is too high, it is possible to either select a substitute substance for that component (e.g. a raw material from another manufacturer) which possibly has a lower peroxide number, or to subject the formulation component concerned to a treatment suitable for reducing the peroxide content. To this end, a treatment with reducing agents is taken into consideration, for example with an inorganic sulfite or hydrogen sulfite, preferably with sodium sulfite or sodium hydrogen sulfite, in each case in aqueous solution (e.g. 5 to 30%-wt). For this treatment, the above-mentioned aqueous solution of the reductive agent is added to the formulation component concerned in an alcoholic solution, preferably in methanolic or ethanolic solution. Through this treatment the peroxides present are readily destroyed in a quick reaction.

Depending on the dissolving properties of the component to be treated, the latter may also be dissolved in an aqueous solution, or in an alcohol-water mixture. If the formulation component or auxiliary substance is a liquid (e.g. a solvent) the treatment can be carried out in such a manner that an aqueous solution of the reducing agent (e.g. sodium sulfite) is added directly to the liquid.

The use of sodium sulfite or sodium hydrogen sulfite is especially advantageous since these substances are pharmaceutically acceptable auxiliaries so that later separation is not necessary.

If precipitation of reaction products occurs, the latter can be separated by centrifugation, sedimentation or filtration.

Following this treatment, the materials are practically free of peroxides and can be employed without hesitation even where the load was previously substantial. An additional improvement of the stability can be attained by using antioxidants, which suppress, or slow down, the formation of new peroxides during the storage of the systems.

The invention and its advantageous properties will be illustrated by means of the following examples.

Film-like preparations were prepared according to the following recipes:

### EXAMPLE 1

| Ethanol/water | |
|---|---|
| Nicotine | 15% |
| HPMC | 79.998% |
| Menthol | 5% |
| Ascorbylpalmitate | 0.002% |

### EXAMPLE 2

| Ethanol/water | |
|---|---|
| Nicotine | 15% |
| HPMC | 79.99% |
| Menthol | 5% |
| Sodium disulfite | 0.01% |

### EXAMPLE 3

| Ethanol/water | |
|---|---|
| Nicotine | 15% |
| HPMC | 79.95% |
| Menthol | 5% |
| Vitamin E | 0.05% |

### COMPARATIVE EXAMPLE

Like Example 1, but without ascorbylpalmitate.

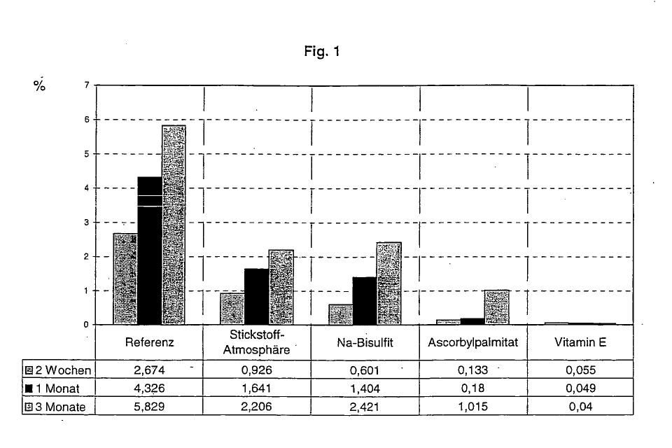

Referring now to FIG. 1, film-like preparations of the compositions indicated in the examples were subjected to a stability test. In this test, the films were stored at 40° C. and a relative air humidity of 75%, and the reduction in the active agent content caused by oxidative processes was determined at certain time intervals (2 weeks, 1 month, 3 months). The results are shown in FIG. 1. The percentage values indicate the content of degradation products, relative to the content of active substance (agent).

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A film-like, active substance-containing preparation for application in the oral cavity or for transmucosal application, wherein said preparation has a maximum peroxide number of 40.

2. The preparation according to claim 1, wherein said preparation has a maximum peroxide number of 15.

3. The preparation according to claim 1, wherein said preparation is substantially free of active oxygen, wherein active oxygen comprises both molecular oxygen and oxygen-containing compounds, wherein the oxygen has an oxidation state higher than −2, and includes peroxides with the general structure R—O—O—R′, wherein R and R′ are selected from the group consisting of alkyl residues and hydrogen, and wherein R and R′ are the same or different.

4. The preparation according to claim 1, wherein said preparation contains at least one antioxidant, selected from the group consisting of ascorbic acid, ascorbylpalmitate, sodium sulfite, sodium disulfite, sodium metabisulfite, tocopherols (vitamin E), tocopherol acetate, thioglycerol, thioglycol acid, vitamin A, propyl gallate, octyl gallate, butylhydroxyanisol and butylhydroxytoluene.

5. The preparation according to claim 4, wherein the concentration of the at least one antioxidant is 0.001 to 5%-wt.

6. The preparation according to claim 1, wherein said preparation comprises a mono-layered or multi-layered polymer matrix, with at least one layer having an active substance content.

7. The preparation according to claim 6, wherein the matrix contains at least one polymer selected from the group consisting of cellulose ether, cellulose acetate, polyvinyl alcohols, polyvinyl acetate, polyvinyl pyrrolidone, polyethylene oxide polymers, polyurethane, polyacrylic acid, polyacrylates, polymethacrylates, alginates, pectins, gelatine, starch and natural rubbers.

8. The preparation according to claim 6, wherein the matrix contains at least one polymer selected from the group consisting of hydrophile, water-soluble polymers and polymers degradable in an aqueous media.

9. The preparation according to claim 1, wherein said preparation has at least one layer or at least one surface, and said at least one layer or at least one surface of the preparation has mucoadhesive properties.

10. The preparation according to claim 1 wherein said preparation contains at least one additive selected from the group consisting of plasticizers, dyes and pigments, degradation enhancers, wetting agents, absorption- or permeation-enhancing substances, pH regulators, fillers, flavouring and aromatic substances and sweeteners.

11. The preparation according to claim 1, wherein said preparation contains at least one active substance being susceptible to attack by peroxide radicals due to the chemical structure of said at least one active substance.

12. Use of a preparation according to claim 1 for transmucosal administration of medicinal active substances by application in the oral cavity.

13. Use of a preparation according to claim 1 as an oral administration form for releasing active substances in the gastrointestinal tract.

14. Use of a preparation according to claim 1 for releasing flavouring or aromatic substances in the oral cavity.

15. A process for the production of a film-like active substance-containing preparation for application in the oral cavity or for transmucosal application, comprising the steps of:
Determining the peroxide number of every formulation component provided for making the preparation of a film-like, active substance-containing preparation for application in the oral cavity or for transmucosal applications wherein the preparation has a maximum peroxide number of 40;
selecting the formulation components wherein the maximum sum of the peroxide numbers of the individual formulation components is 40, with the peroxide number of each formulation component being weighted according to the percentage of these components in the preparation;
preparing a solution, dispersion or melt containing the selected formulation components and at least one active substance to be released;
coating the solution, dispersion or melt onto an inert support using doctor-knife application, roll application, spraying or extrusion methods; and drying or cooling said preparation to form a film layer.

16. The process according to claim 15, wherein the maximum sum of the peroxide number is 15.

17. The process according to claim 15, and further comprising the step of subjecting at least one formulation component to a treatment with at least one reducing agent for reducing the peroxide content.

18. The process according to claim 17, and further comprising the step of adding an aqueous solution of an inorganic sulfite salt or hydrogen sulfite salt to the formulation component in an alcoholic solution.

19. The preparation according to claim 2, wherein said preparation has a maximum peroxide number of 5.

20. The preparation according to claim 5, wherein the concentration of the at least one antioxidant is 0.01 to 3%-wt.

21. The preparation according to claim 7, wherein said cellulose ether is at least one selected from the group consisting of ethyl cellulose, propyl cellulose, carboxymethyl cellulose (CMC), hydroxypropyl cellulose (HPC) and hydroxypropylmethyl cellulose (HPMC).

22. The preparation according to claim 8, wherein said at least one polymer is selected from the group consisting of cellulose derivatives, polyvinyl alcohol, polyvinyl acetate, polyvinylpyrrolidone, polyacrylates, water-soluble polysaccharides and proteins.

23. The preparation according to claim 22, wherein said cellulose derivatives are selected from the group consisting of hydroxypropylmethyl cellulose, carboxymethyl cellulose, hydroxypropyl cellulose and methyl cellulose.

24. The preparation according to claim 22, wherein said water-soluble polysaccharides are selected from the group consisting of pullulan, xanthan, alginates, dextrane and pectins.

25. The preparation according to claim 22, wherein said proteins are gel-forming proteins.

26. The preparation according to claim 25, wherein said gel-forming proteins are gelatine.

27. The process according to claim 16, wherein the maximum sum of the peroxide number is 5.

28. The process according to claim 18, wherein said inorganic sulfite salt or hydrogen sulfite salt is selected from the group consisting of sodium sulfite and sodium hydrogen sulfite.

29. The process according to claim 18, wherein said alcoholic solution is selected from the group consisting of a methanolic solution and an ethanolic solution.
